(19) United States
(12) Patent Application Publication
(10) Pub. No.: US 2002/0146744 A1
(43) Pub. Date: Oct. 10, 2002

(54) Combinatorial chemistry and compound identification system

(76) Inventors: Nabil M. Lawandy, North Kingstown, RI (US); Joseph M. Calo, Greenville, RI (US)

Correspondence Address: HARRINGTON & SMITH, LLP, 4 RESEARCH DRIVE, SHELTON, CT, 06484-6212, US

(21) Appl. No.: 10/087,525

(22) Filed: Mar. 1, 2002

Related U.S. Application Data
(60) Provisional application No. 60/273,188, filed on Mar. 2, 2001.

(51) Int. Cl. G01N 33/53; C12M 1/34; G01N 33/543

(52) U.S. Cl. 435/7.1; 436/518; 435/287.1

(57) ABSTRACT

A combinatorial chemistry system (100) uses a set of fluidized bed reactors (1) and spectrographically unique compound growth structures (2) for subsequent identification of compounds. A method for identification of compounds manufactured in the combinatorial chemistry system is also disclosed.

### CLAIM OF PRIORITY

This patent application claims priority under 35 U.S.C §119(e) from copending U.S. Provisional Patent Application No.: 60/273,188, filed Mar. 2, 2001.

### REFERENCE TO A RELATED PATENT APPLICATION

Incorporated by reference herein is a pending U.S. patent application Ser. No. 09/310,825, filed May 12, 1999, entitled “Micro Lasing Beads and Structures for Combinatorial Chemistry and Other Applications, and Techniques for Fabricating the Structures and for Detecting Information Encoded by the Structures,” which claims priority from U.S. Provisional Applications No. 60/085,286 filed May 13, 1998; No. 60/086,126 filed May 20, 1998, No. 60/127,170 filed Mar. 30, 1999; and No. 60/128,118 filed Apr. 7, 1999. U.S. patent application Ser. No. 09/310,825, filed May 12, 1999, is incorporated by reference herein in its entirety.

### TECHNICAL FIELD

These teachings relate generally to a system and a method for encoding and decoding information useful in a combinatorial chemistry system for the synthesis and identification of newly formed compounds.

### BACKGROUND

The early steps of drug discovery are reliant upon a variety of factors. Creating drugs to address a specific problem has required, among other things, knowledge of biochemical mechanisms and processes, as well as the design and manufacture of what have been typically large arrays of compounds. Once these arrays of chemical compounds have been created, experimentation has ensued to test candidate compounds for efficacy. Historically, creating these large arrays, or libraries, of compounds has been time consuming and expensive. Recent advances in various technologies have provided for improvements in the process of creating a library of chemical compounds. One of the most notable advances may be the introduction of combinatorial chemistry systems.

In a typical combinatorial chemistry system, a designated set of reagents is used to produce a comparatively large number of experimental compounds. First, an experimenter will determine a number of reagents that have a potential to form a desired type of compound. Once the reagents have been identified, they are introduced into an automated system. The automated system then progressively combines the reagents in a manner that is dictated by the needs of the experiment. Consider, for example, the process of mixing two sets of chemicals, each set being comprised of three unique chemicals. When each of three chemicals of one set are mixed with each element of the other set, nine unique combinations are possible.

Sophisticated combinatorial chemistry systems provide a number of advantages over manual methods for the synthesis of experimental compounds. For example, automated systems provide for a high degree of reproducibility and control in the experimental process in comparison to traditional manual methods. This inevitably has led to the ability to synthesize large numbers of compounds, thereby accelerating discovery, saving time, money, and creating smaller amounts of waste. In addition, automated systems have provided users with the ability to create sophisticated combinations under a variety of experimental conditions.

One problem with combinatorial chemistry systems is the accurate identification of the formula for the variety of newly formed compounds. The use of bar coding and other similar schemes provide for automation, but these systems are not as accurate or as flexible as needed to support many types of experiments.

One feature of current combinatorial chemistry technology is the use of a large number of so-called solid supports or beads as a matrix or growth matrix phase. These solid supports or structures (herein also referred to as beads) are used to provide a support surface to which the new compounds bonded. Although the use of beads has a number of experimental benefits, such benefits are not relevant here. However, the presence of these beads is significant for the improvements to combinatorial chemistry disclosed herein.

Reference can be had to WO 96/36436, “Remotely Programmable Matrices with Memories and Uses Thereof”, Nova et al. and to U.S. Pat. No.: 6,096,496, “Supports Incorporating Vertical Cavity Emitting Lasers and Tracking Apparatus for Use in Combinatorial Synthesis”, by Frankel, in particular the Scatter Medium Laser (SML) embodiments. Reference can also be made to U.S. Pat. No.: 5,448,582, “Optical Sources Having a Strongly Scattering Gain Medium Providing Laser-Like Action”, by Lawandy, as well as to divisions thereof found in U.S. Pat. Nos. 5,625,456 and 5,825,790, incorporated by reference herein in their entireties.

### SUMMARY OF THE PREFERRED EMBODIMENTS

The foregoing and other problems are overcome, and other advantages are realized, in accordance with the presently preferred embodiments of these teachings.

This invention provides a novel encoding and decoding system for drug discovery and other important applications. More specifically, the present invention includes a system for reacting a sample or library of samples with reusable and encoded carrier units under controlled conditions, and thereafter for identifying, at least for analysis purposes, the encoded carrier units, also referred to herein as beads or as growth matrix containing structures.

This invention employs a matrix growth structure, and techniques for use of the matrix growth structure, such as the one or ones taught by the above-referenced U.S. patent application Ser. No. 09/310,825, filed May 12, 1999, “Micro Lasing Beads and Structures for Combinatorial Chemistry and Other Applications, and Techniques for Fabricating the Structures and for Detecting Information Encoded by the Structures.”

For simplicity, the spectrographically unique matrix growth structures described in the referenced patent application may be referred to herein individually as a LaserChip™ or a LaserBead™, or more simply as a “bead” or as a growth matrix containing structure.

In the presently preferred embodiment a set of fluidized bed reactors is operated through more than one cycle to create multiple compounds on a plurality of beads. Experimental factors, among other things, determine the number and nature of the reactors, the number and nature of the reactor cycles, the reagents used, the character of the beads, and other factors that will affect the synthesis of chemical compounds.

In one embodiment a combinatorial chemistry system includes the set of fluidized bed reactors. A number of randomly distributed and spectrographically unique structures or beads are introduced into each reactor and different reagents are introduced into each reactor. Thereafter, each reactor is operated for a specified time under appropriate conditions to circulate the reagent over the beads and to mix the beads and the reagent, and is then shut down. Once shutdown, the reagent and the beads are dispensed under computer control from each reactor in turn and directed as a fluidized steam of beads through a bead reader. The bead reader, which is capable of detecting the unique spectrographic signature of each bead, reads the spectrographic signature of each bead and records information identifying the bead, as well as the reactor from which the bead originated. The beads are then sent to a single collection bin where they are washed and mixed in a fluidized environment. The set of reactors is then prepared for a another cycle with appropriate cleaning or other preparations suited to the experimental situation.

Following the washing and mixing of the beads and the preparation of the reactors, the quantity of prepared beads are divided, preferably more or less evenly, and again randomly dispensed into the series of reactors. The division can be done by simply weighing out approximately equal amounts of beads into a plurality n of weight sets, where n is the number of fluidized bed reactors, and placing a weight set of beads into one reactor. A second set of reagents is dispensed into the set of reactors. After the reactors have again cycled appropriately, the process of emptying the reactors and directing a fluidized steam of the beads through the bead reader is initiated. Consistent with the first cycle, the reader identifies each bead and associates the bead with the reactor from which the bead originated. As with the first cycle, the beads are automatically sent to a single collection bin where they are washed and mixed in a fluidized environment, and readied for use in another cycle, if desired.

After the synthesis steps have been completed, the user may sort the beads as needed for further experimentation, or proceed in whatever other manner is deemed to be suitable.

As an example of this embodiment, consider 1,000 spectrographically unique beads that are introduced into 10 fluidized bed reactors, approximately 100 beads being introduced into each reactor, and then processed through two reactor cycles.

In the first cycle, 10 unique reagents (referred to in this example as reagents 1 through 10 for the first reactor cycle) are dispensed, one reagent into each reactor. After the reactors have completed operation, the contents of each reactor are directed through the bead reader. The reader individually identifies each bead, associates each individual bead with a specific reactor, and submits appropriate information to a database. The beads from each reactor are directed to a single collection bin, where the 1,000 beads are washed and thoroughly mixed.

A quantity of approximately 100 of the 1,000 beads, each one carrying one of the reagents 1 through 10, is introduced into each reactor. Consistent with the first cycle, a set of unique reagents (referred to in this example as reagents A through J for the second cycle) is then introduced into the system, and a (preferably) different reagent is dispensed into each reactor. The reactors are fluidized and operated so as to throughly mix the beads with the reagent, and after cycling of the reactors has completed, the contents of each reactor are again directed through the reader. The reader again identifies each spectrographically unique bead, and associates each bead with the specific reactor from which it was just extracted. This process may be repeated any number of times.

In this example the original quantity of 1,000 beads can carry 100 different compounds. These compounds are formed from reagents 1 through 10, and reagents A through J.

The use of the present invention can be for the following exemplary applications: library production, chemical optimization, lead optimization (focused libraries) and chemical development.

The combinatorial chemistry system of this invention is particularly well suited for mix and split, or split and mix, or pool and split applications, and can also be used for parallel or high throughput synthesis applications. Increased time efficiency and reduced reagent requirements are achievable (relative to parallel synthesis). The system of this invention also provides rapid and precise decoding of the solid support (bead) and the attached compound, and provides an ability to synthesize compounds in a broad chemical space.

### BRIEF DESCRIPTION OF THE DRAWINGS

The foregoing and other aspects of these teachings are made more evident in the following Detailed Description of the Preferred Embodiments, when read in conjunction with the attached Drawing Figures, wherein:

FIG. 1 is an illustration of components of a combinatorial chemistry system that uses beads for encoding and decoding of compound information.

FIG. 2 is an illustration of the combinatorial chemistry system described in FIG. 1, wherein the system is designed to support manual cleaning and charging.

FIG. 3 is an illustration of the relative size of a Bead designed for use in a combinatorial chemistry system.

FIG. 4 is an illustration of a hydrodynamic-based reader, also known as a bead emission reader.

FIG. 5 is an illustration of a single fluidized bed reactor, where the fluidized bed reactor is charged with beads and a reagent.

### DETAILED DESCRIPTION OF THE PREFERRED EMBODIMENTS

In a preferred embodiment of this invention beads that are supportive of optical encoding processes are used as a matrix growth structure for development of chemical compounds. These beads, and optical techniques for use of these beads, are described in detail in the U.S. patent application Ser. No. 09/310,825, filed May 12, 1999, entitled “Micro Lasing Beads and Structures for Combinatorial Chemistry and Other Applications, and Techniques for Fabricating the Structures and for Detecting Information Encoded by the Structures,” incorporated by reference herein in its entirety. However, in other embodiments other types of beads can be used, including beads that contain active light emitting components such as LEDs or laser diodes.

It should be realized that the teachings of this invention could be employed in a variety of combinatorial chemistry systems. In the presently preferred, but non-limiting embodiment, the combinatorial chemistry system uses fluidized bed reactors (FBRs) to mix the beads with selected reagents.

In a preferred embodiment, beads are used with a set of FBRs to create a combinatorial chemistry system for the generation and identification tracking of new and unique compounds.

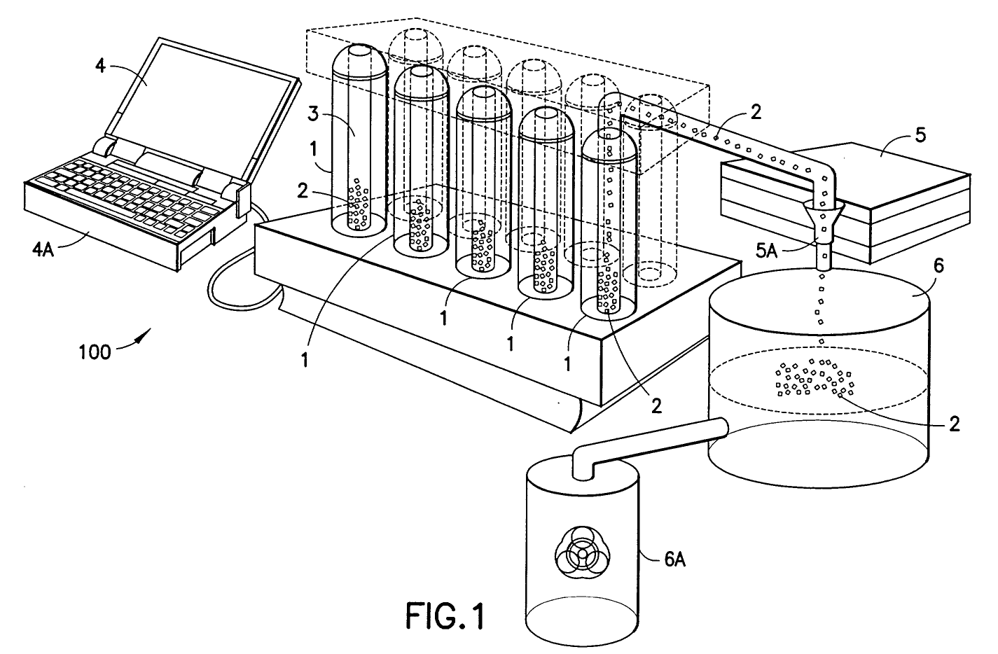

FIG. 1 illustrates components of a combinatorial chemistry system 100 that uses beads 2 for encoding and decoding of compound information. In this embodiment, a set of 10 FBRs 1 is used. Each of the FBRs 1 is charged with a quantity of spectrographically individually unique beads 2. Subsequently, a quantity of reagent 3 is introduced into each of the FBRs 1. Each FBR 1 is then operated in a manner that is consistent with the needs of the process. Operation of the FBR 1 serves to coat each of the beads 2 with a quantity of reagent 3.

Once the beads 2 in each FBR 1 have been prepared with a reagent 3, the FBR 1 contents containing reagent 3 and beads 2 are emptied either automatically or manually. In the arrangement where the emptying occurs automatically, a system controller, such as a computer 4 running appropriate software 4A, initiates flow of the contents of each FBR 1 in a sequential manner. Once flow of the contents of each FBR 1 has been initiated, the contents are directed through a reader station 5.

In a preferred embodiment the reader station 5 illuminates each bead 2 and identifies the spectrographic signature of each Bead 2. The contents of the FBR 1 are then directed from the reader station 5 to a collection bin 6. The contents of the set of FBRs 1 in the combinatorial chemistry system 100 are progressively emptied into the collection bin 6 in this manner, while each bead 2 is passed through the reader 5 and its spectrally unique signature detected and recorded, in association with an identification of the specific one of the FBRs 1 from which it was just extracted. All of this information can be recorded and saved by the computer 4, which is also assumed to have a record of which reagent(s) were used in each of the FBRs 1.

The contents so deposited into the collection bin 6 are washed and mixed with the contents of the other FBRs 1 in the combinatorial chemistry system 100. The washed and mixed contents are set aside for use in a subsequent cycle of the FBRs 1.

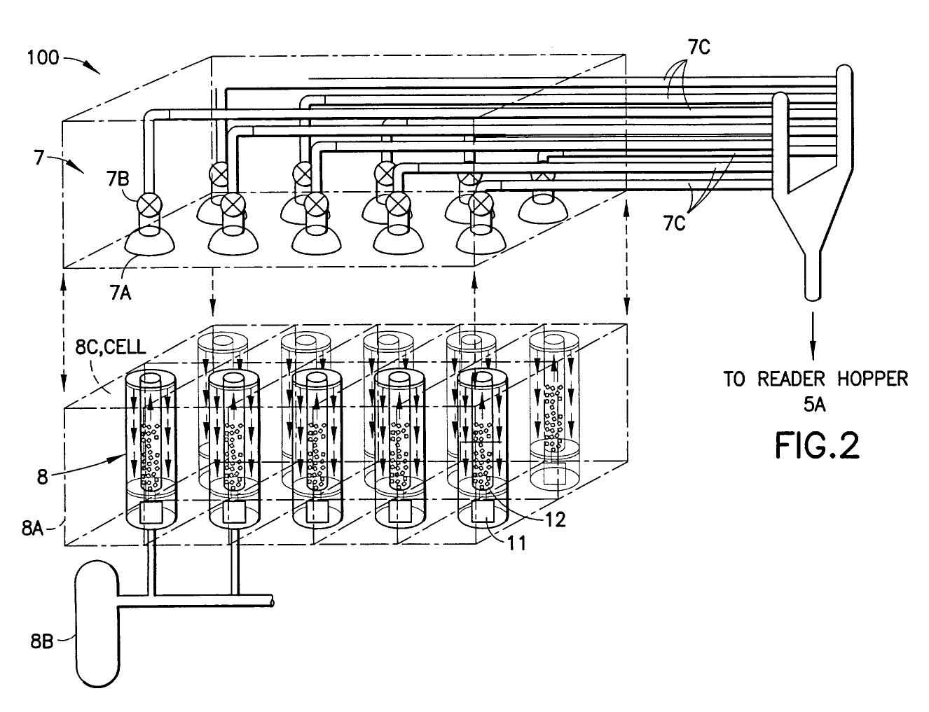

At this point the combinatorial chemistry system 100 including the FBRs 1 can be cleaned and prepared for the next cycle. FIG. 2 shows the manual separation of the FBRs 1. Separation of an upper manifold compartment 7 from the FBR vessels 8 permits a user to clean the reactor internals with appropriate means including, but not limited to, the use of solvents, soaps and heat. A waste tank 6A can be provided for collecting used reagents as well as cleaning materials.

Once cleaning of the combinatorial chemistry system 100 has been completed, the washed and mixed beads 2 produced by the first cycle can be approximately evenly distributed, such as by weight or by volume, and deposited within each FBR vessel 8. Each FBR vessel 8 is then manually or automatically charged with reagent and the upper manifold compartment 7 is then coupled to the FBR vessel 8. Once reassembly of the FBRs 1 has been completed, the FBRs 1 are operated for a second cycle. The order of filling the reactor vessels 8 could be reversed such that the reagent(s) are added first followed by the beads 2.

More specifically, the bottom reactor compartment or reactor box 8A holds, for example, 10 fluidized bed reactors 1, each in its own individual thermostated cell 8C. Each reactor vessel 8 is fixed in place and plumbed to a central solvent reservoir 8B which is used for cleaning the reactors between reaction cycles. Representative, but limiting, dimensions for one of the reactors 1 is an inside diameter of about 1.5 inches and a height of about 8 inches. The reactors 1 can be comprised of any suitable, non reactive material, such as glass or stainless steel.

The upper manifold compartment 7 or manifold box holds in place individual, O-ringed mating flanges 7A for each reactor and the manifold system including valves 7B and piping 7C for transport of the beads 2 to the reader station 5.

Following a reaction run, the top and bottom compartments are manually or automatically separated for cleaning out the reactors 1 with solvent, and recharging them with the next batch of beads 2. Once the reactors 1 are all charged with beads 2, they are each (manually or automatically) charged with the appropriate reaction medium, the top manifold compartment 7 is fixed in place and clamped to the bottom compartment 8A to O-ring seal the reactor flanges 7A, and the reaction sequence is initiated.

Following the reaction run, the beads 2 from each reactor are sequentially entrained with a liquid, such as a solvent and/or the reagent, by activating each valve 7B in a programmed fashion. The beads 2 are convected to the reader hopper 5A through individual fluid lines 7C connected to reactor compartments 8 through the valves 7B.

The process employed in the first cycle to collect the beads 2 from the FBRs 1 is again used for the second cycle. That is, once the beads 2 in each FBR 1 have been prepared with a second reagent 3, a system controller, such as the computer 4 running appropriate software 4A, initiates flow of the contents of each FBR 1 in a progressive manner. The contents containing reagent 3 and beads 2 are directed through the reader station 5.

The reader station 5 illuminates each bead 2 and detects the spectrographic signature of each bead 2. The contents of the FBR 1 are then directed from the reader station 5 to the single collection bin 6. The contents of the set of FBRs 1 in the combinatorial chemistry system 100 are progressively emptied into the single collection bin 6 in this manner. The contents so deposited into the collection bin 6 are washed and mixed with the contents of the other FBRs 1 in the system.

In this embodiment, the beads 2 that have been processed through two cycles may host a variety of unique compounds. For example, if ten unique reagents 3 are used in the first cycle and another ten unique reagents 3 are used in the second cycle, one hundred unique compounds might be formed. Once synthesized, these compounds may either be subjected to a continuation of compound synthesis steps, used for experimentation, or other disposition as deemed suitable by the experimenter.

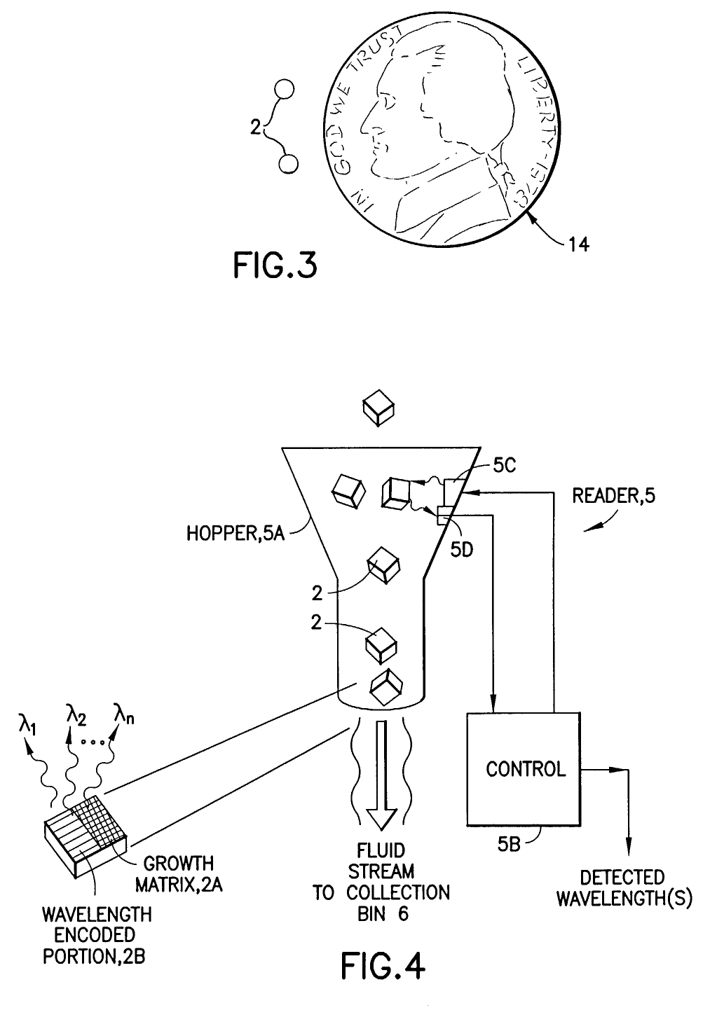

FIG. 3 illustrates the relative size of a bead 2. In FIG. 3, two beads 2 are shown alongside a coin 14.

In general, the beads 2 may be read at a high rate, such as at a rate of 60 beads/second while being transported in a fluid environment through the reader station 5. The beads 2 can be read with a high degree of accuracy (e.g., error rate of less than 1/million). In one embodiment each bead 2 can be encoded such that there may be up to about 1,000 unique codes per bead. Due to the robustness of the optical reading procedure the beads 2 can be accurately read even when the codes are partially obscured, and they can be read in any orientation (omnidirectional). In the presently preferred, but not limiting embodiment, each bead 2 can accommodate about 1-5 mgs of compound loading (size 5×5×2 mm). The beads 2 are stable under a wide range of environmental conditions (e.g., solvent, temperature, suspended solids, photo-cleavage). In the preferred embodiment the reading of the stimulus and ID spectral signatures does not significantly interfere with or cause damage to the attached molecules, and the robustness has been validated in peptide synthesis.

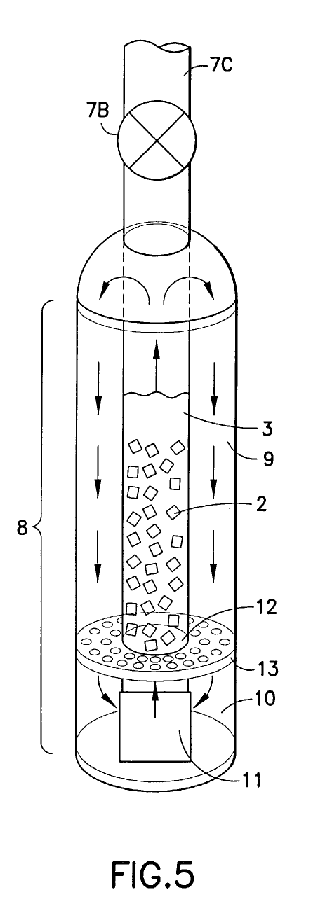

FIG. 4 is an illustration of the hydrodynamic reader station 5. A fluid stream containing beads 2 is introduced through one of the lines 7C to the reader hopper 5A. As shown in the enlarged view, each bead 2 can contain a growth matrix portion 2A wherein the reagents may react to form more complex molecules. The growth matrix portion 2A could comprise any one of a plurality of commercially available resins, or it could comprise a polymer-grafted surface. Each bead 2 can also contain a wavelength encoded portion 2B containing a plurality of discrete areas, each capable of emitting a characteristic wavelength (lambda_1 through lambda_n). The set of wavelengths uniquely identifies the bead 2. Disposed in or near the hopper 5A is a light source 5C, such as a LED, a laser diode, a flashlamp, or any suitable light source for exciting the fluorescent or phosphorescent material contained in the wavelength encoded portion 2B to emit the characteristic wavelengths. The emitting material could also be capable of emitting a laser-like emission, such as described in the above-referenced U.S. Pat. No. 5,448,582, “Optical Sources Having a Strongly Scattering Gain Medium Providing Laser-Like Action”, by Lawandy. Also disposed in or near the hopper 5A is a multi-spectral detector 5D. The detector 5D may be constructed using a plurality of photodetectors each having an associated passband filter (corresponding to lambda_1 through lambda_n). Alternatively, it could be constructed using an area detector placed behind a wedge or other type of wavelength dispersing filter. Alternatively, the detector 5D could be comprised of a plurality of discrete photodiodes, each being constructed and bandgap tuned so as to be responsive to a particular relatively narrow band of wavelengths.

A controller 5B, such as an embedded microprocessor, can be provided for controlling the source 5C, reading out the detectors 5D and interfacing with the computer 4. The output of the controller 5B can be an indication of the detected wavelengths, which in turn can be stored in the computer 4 and correlated with the identity of the reactor 1 that is currently being emptied through the hopper 5A.

FIG. 5 is an illustration of one of the FBRs 1. Shown is the orientation of the beads 2 within the FBR 1 and the direction of flow. The reagent 3 circulates down the liquid return 9 of the reactor to a liquid reservoir 10. A liquid pump 11 in the base of the FBR 1 pumps the returned reagent 3 up through a liquid distributor 12. A perforated base or screen 13 separates the liquid reservoir 10 from the upper portion of the reactor vessel 8. The beads 2 are constrained to remain within the liquid distributor 12, which essentially defines a liquid column with a vertical upward flow within the downward flow of the surrounding liquid return column 9. The FBR 1 is emptied when the valve 7B is opened, either manually or automatically under control of computer 4, and the contents, including the fluidized beads 2 and reagent 3, and possibly a solvent or even water, are directed from the FBR 1 to the reader hopper 5A via one of the pipes 7C, as described above.

It should be noted that it is within the scope of these teachings to control the density of the beads 2, such as by adding/removing weight. In this manner, and as examples, the bead weight could also be used as a combinatorial variable, or for separation of beads within fluidized bed reactor 1, or for exposing certain of the beads 2 to selective reaction conditions within the FBR 1. In a similar fashion, control or modification of the fluidizing medium (that can be or include an aqueous solution) can also be used to accomplish some of these same objectives. For example, the density of water can be decreased by adding polymer microbubbles, or the density can be increased by using additives such as finely ground magnetite. This a distinctive feature of FBRs that can be exploited to advantage in the combinatorial chemistry system 100 in accordance with the teachings of this invention.

The feature of independent temperature control of each FBR 1 is also an important characteristic, as the temperature can also be used as a combinatorial variable. This is an advance over conventional “well-plate” systems.

As such thus be apparent, the combinatorial chemistry system 100 of this invention is particularly well suited for mix and split, or split and mix, or pool and split combinatorial chemistry applications, and can also be used for parallel or high throughput synthesis applications.

Although described in the context of presently preferred embodiments, those skilled in the art should appreciate that a number of changes to the overall form and details of these embodiments may be made, and that the resulting modified system and methods will still fall within the scope of this invention. For example, more or less than 10 FBRs 1 can be employed. Furthermore, other than optically-based bead identification techniques may be used in some embodiments, such as one based on radio frequency identification (RF ID). In this case the reader station 5 can include a source of RF or optical energy for stimulating the RF ID beads to transmit their encoded identification information. Note as well that in some embodiments it may be desirable to incorporate the data processing and data storage capabilities of the computer 4, including any automatic control over the pumps 11, valves 7B and the like, into the reader station 5.

What is claimed is:

1. A method for operating a combinatorial chemistry system using growth matrix containing structures supportive of an identification encoding technique, comprising:
placing said structures and a reagent into at least one fluidized bed reactor;
operating said at least one fluidized bed reactor to circulate said reagent over said structures;
directing said reagent and said structures entrained within said reagent to a reader station;
uniquely identifying individual ones of said structures using said reader station; and
recording an identity of said structures in association with an identification of the reagent.

2. A method as in claim 1, further comprising:
directing said structures to a collection bin; and
washing said structures prior to reuse of said structures, and continuing the method until said structures have been exposed to a desired plurality of reagents.

3. A method as in claim 1, where said structure emits a signal or signals that identify said structure in response to excitation energy applied by said reader station.

4. A method as in claim 1, where said structure emits a plurality of optical wavelengths that identify said structure in response to excitation energy applied by said reader station.

5. A method as in claim 1, where there are a plurality of said fluidized bed reactors all capable of simultaneous operation, and where at least two of said plurality of fluidized bed reactors contain different reagents.

6. A method as in claim 1, where said step of operating said at least one fluidized bed reactor to circulate said reagent over said structures comprises constraining said structures to remain within a liquid column with a vertical upward flow that is contained within a downward flow of a surrounding liquid return column.

7. A combinatorial chemistry system that uses growth matrix containing structures supportive of an identification encoding technique, comprising:
a set of fluidized bed reactors individual ones of which are for containing a quantity of said structures and a reagent, and operating to circulate said reagent over said structures;
a reader station for selectively coupling to individual ones of said set of fluidized bed reactors through fluid communication means, said reader station operating to uniquely identify individual ones of said structures as they pass through said reader station; and
a data processor for recording an identity of said structures in association with an identification of the reagent.

8. A system as in claim 7, further comprising a collection bin downstream from said reader station wherein washing of said structures occurs prior to reuse of said structures.

9. A system as in claim 7, where said structure emits a signal or signals that identify said structure in response to excitation energy applied by said reader station.

10. A system as in claim 7, where said structure comprises a material for emitting a plurality of optical wavelengths that identify said structure in response to excitation energy applied by said reader station.

11. A system as in claim 7, where at least two of said plurality of fluidized bed reactors contain different reagents.

12. A system as in claim 7, where each of said fluidized bed reactors operates to constrain said structures to remain within a liquid column with a vertical upward flow that is contained within a downward flow of a surrounding liquid return column.

13. A system as in claim 7, where said set of fluidized bed reactors is comprised of a lower set of reactor vessels and an upper set of reactor vessel flanges.

14. A system as in claim 7, where said set of fluidized bed reactors is comprised of a bottom reactor box that holds a set of n fluidized bed reactor vessels and an upper manifold box that holds in place a set of n individual mating flanges comprising sealing means, as well as a manifold system comprising n valves and n pipes for transport of said structures to said reader station.

15. A system as in claim 14, where said bottom reactor box and said upper manifold box are manually separable form one another and manually joinable to one another.

16. A combinatorial chemistry system operable with a set of growth matrix containing structures, comprising:
a set of fluidized bed reactors individual ones of which are for containing a quantity of said structures and a reagent, and operating to circulate said reagent over said structures;
a reader station for selectively coupling to individual ones of said set of fluidized bed reactors through fluid communication means, said reader station operating to uniquely identify individual ones of said structures as they pass through said reader station by detecting a set of optical wavelengths emitted by each structure, where the set of optical wavelengths uniquely identifies said structure within said set of structures; and
a data processor for recording an identity of said structures in association with an identification of the reagent.

17. A system as in claim 16, further comprising a collection bin downstream from said reader station wherein washing of said structures occurs prior to reuse of said structures.

18. A system as in claim 16, where said structure comprises a material for emitting said set of optical wavelengths in response to excitation energy applied by said reader station.

19. A system as in claim 16, where each of said fluidized bed reactors operates to constrain said structures to remain within a liquid column with a vertical upward flow that is contained within a downward flow of a surrounding liquid return column.

20. A system as in claim 16, where said set of fluidized bed reactors is comprised of a bottom reactor box that holds a set of n fluidized bed reactor vessels and an upper manifold box that holds in place a set of n individual mating flanges comprising sealing means, as well as a manifold system comprising n valves and n pipes for transport of said structures to said reader station, and where said bottom reactor box and said upper manifold box are manually separable form one another and manually joinable to one another such that individual ones of said n fluidized bed reactor vessels are simultaneously joined with and sealed to said set of n individual mating flanges.

21. A method for operating a combinatorial chemistry system using growth matrix containing structures supportive of an identification encoding technique, comprising:
placing said structures and a fluidizing medium comprising a reagent into at least one fluidized bed reactor;
operating said at least one fluidized bed reactor in accordance with at least one combinatorial variable for mixing said structures with said reagent;
directing said fluidizing medium and said structures entrained within said fluidizing medium to an identification station;
stimulating individual ones of said structures to emit a signal for uniquely identifying individual ones of said structures; and
recording an identity of said structures in association with an identification of the reagent.

22. A method as in claim 21, where said at least one combinatorial variable comprises bead weight.

23. A method as in claim 21, where said at least one combinatorial variable comprises fluidizing medium density.

24. A method as in claim 21, where said at least one combinatorial variable comprises temperature.
